(19) United States
(12) Patent Application Publication
(10) Pub. No.: US 2016/0220587 A1
(43) Pub. Date: Aug. 4, 2016

(54) VITAMIN D COMPLEXES WITH DE-VDBP AND AN UNSATURATED FATTY ACID, AND THEIR USE IN THERAPY

(71) Applicant: David NOAKES, Guernsey, Channel Islands (GB)

(72) Inventor: Marco Ruggiero, Firenze (IT)

(21) Appl. No.: 14/900,249

(22) PCT Filed: Jun. 13, 2014
(86) PCT No.: PCT/GB2014/051824

§ 371 Date: Dec. 21, 2015

(30) Foreign Application Priority Data
Jun. 21, 2013 (GB) 1311134.9
Feb. 27, 2014 (GB) 1403508.3

(51) Int. Cl. A61K 31/593; A61K 47/48; A61K 9/00

(52) U.S. Cl. CPC A61K 31/593; A61K 9/0053; A61K 9/0078; A61K 9/0031; A61K 47/48038; A61K 47/48238

(57) ABSTRACT

A family of potent, stable vitamin D-based complexes for therapeutic use in chronic diseases such as cancer, neuro-degenerative diseases, chronic kidney disease and HIV infection is disclosed. They may be made by interaction with de-glycosylated vitamin D-binding protein to form a dimeric complex which can be further stabilized by unsaturated fatty acids to form a trimeric complex which provides improved interaction at cellular level with the vitamin D receptor at the plasma membrane. Effectiveness can be further improved by dissolving or solubilizing the supplement in a suitable solvent. One delivery mechanism for the complexes is to encapsulate them in liposomes, enabling oral administration.

### TERMINOLOGY

In the description which follows, to assist readability, the following terms are used; each being accompanied by its meaning:
VDBP: vitamin D-binding protein.
de-VDBP: vitamin D binding protein de-glycosylated at position Thr (threonine) 420 by sequential removal of sialic acid and galactose.
Vit Ds: vitamin D and its analogues.
VDR: vitamin D receptor.
UFA: unsaturated fatty acids.
Dimeric/trimeric/tetrameric complexes: stable association of 2, 3 or 4 molecules held together by chemical interactions between specific recognition sites. The molecules that form the complex are indicated inside [square brackets]
Mixtures: mixtures where there are several molecular species that, however, do not bind to each other. For example; “free” de-VDBP+[Vit Ds/de-VDBP]+[Vit Ds/de-VDBP/UFA].

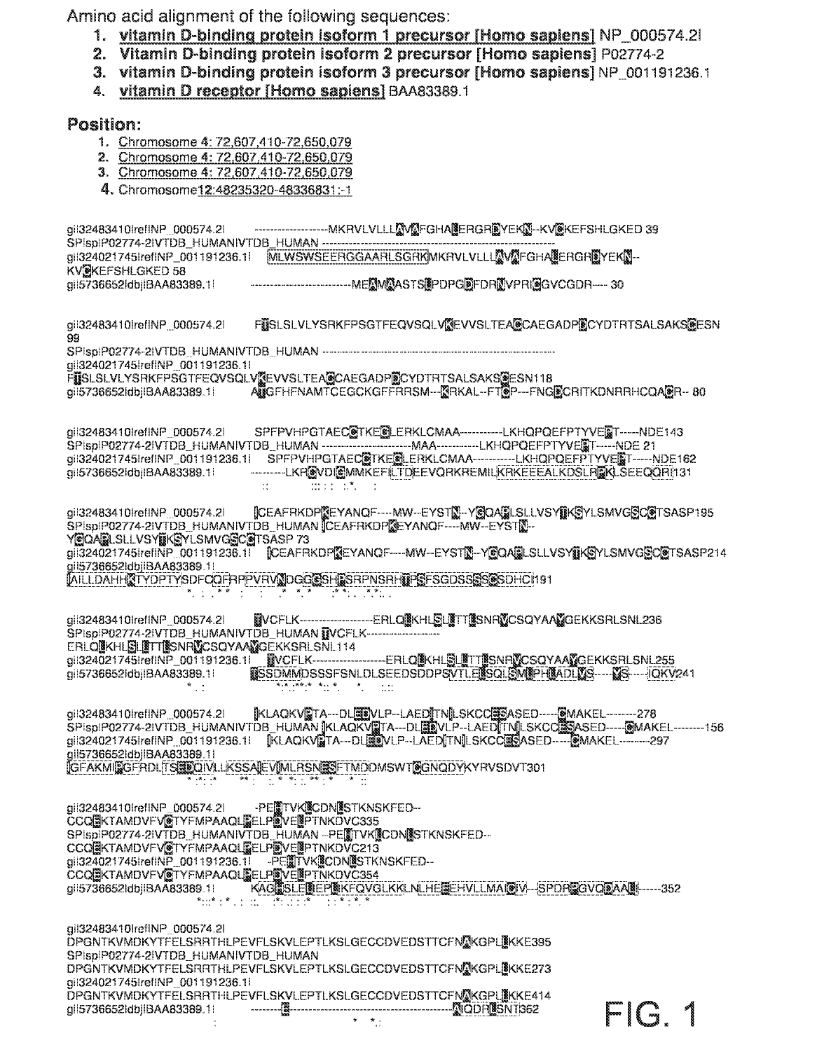

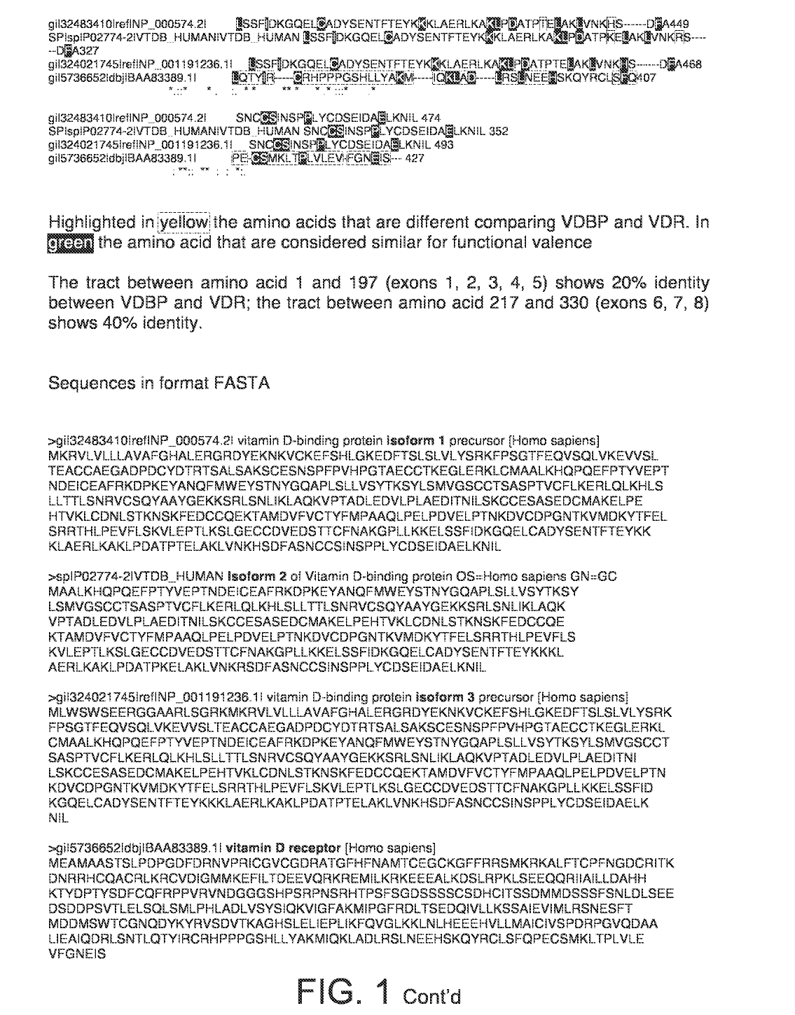

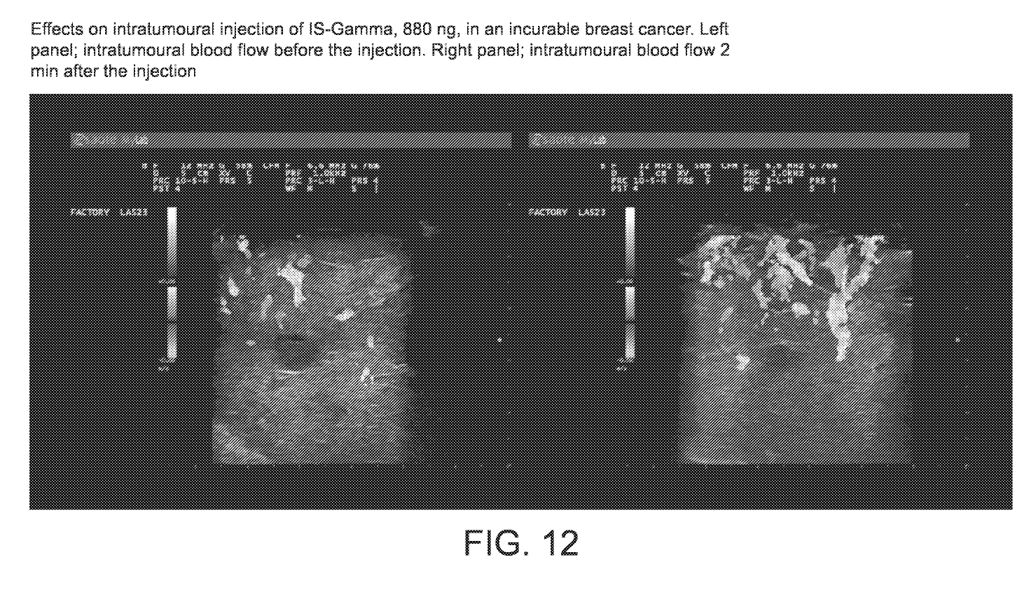

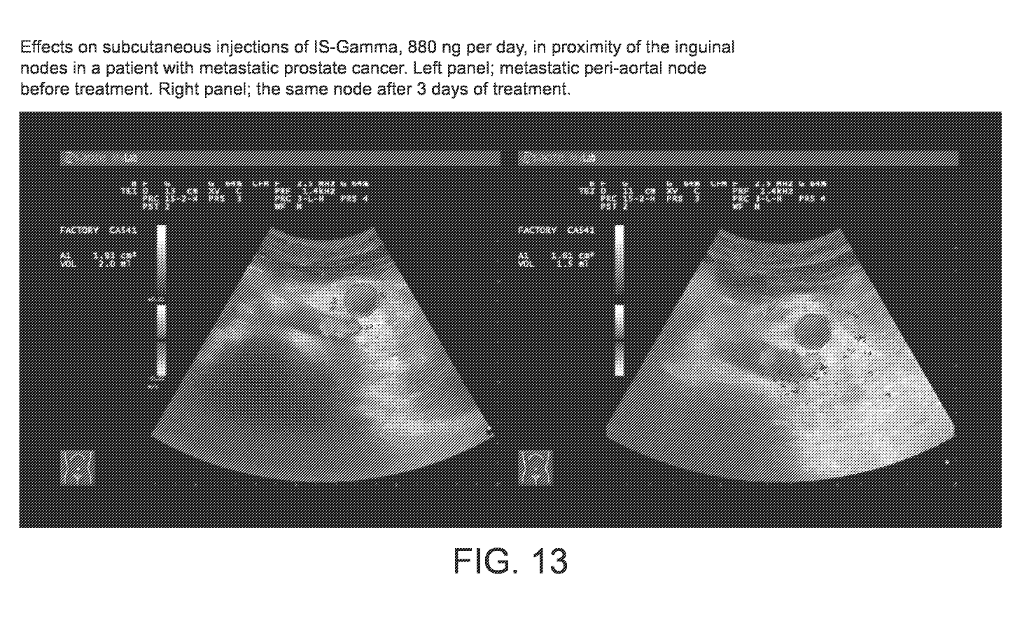

Also in the description which follows, reference is made to the accompanying FIGS. 1 to 11 which form a part of the description of the invention. FIGS. 12 and 13 illustrate the therapeutic effect when the invention is put into practice in a clinical treatment.

### FIELD OF THE INVENTION

This invention relates to vitamin D-based complexes, particularly but not exclusively for use as supplements. In particular it provides a family of potent, stable vitamin D-based supplements for complementary therapeutic usage in chronic diseases such as cancer, neurodegenerative diseases, chronic kidney disease and HIV infection.

The description which follows discloses the preparation of a family of molecules based on the backbone of de-glycosylated vitamin D binding protein (de-VDBP); vitamin D₃ and other vitamin D receptor agonists are bound to such a backbone via hydrophobic interactions. Unsaturated fatty acids are also bound to the de-VDBP backbone to favour interaction with cellular membranes. These stable complexes may be encapsulated in liposomes for oral bioavailability. This family of novel, stabilised complexes may be used in all those conditions where supplementation of vitamin D has proven effective.

### BACKGROUND TO THE INVENTION

Vitamin D and the Vitamin D Axis

There has been much recent interest in the role of the vitamin D axis in a wide variety of chronic diseases. The vitamin D axis includes the active form of vitamin D that is vitamin D₃, the vitamin D receptor (VDR) and the vitamin D-binding protein (VDBP). VDBP exists in different isoforms and a linear O-linked trisaccharide of the type GalNAc-Gal-Sia is attached to the threonine residue at position 420 (Thr 420) in two of the three most common isoforms termed Gc1s and Gc1f (Biochim Biophys Acta. 2010 April; 1804(4):909-17). VDBP can be de-glycosylated by treatment with sialidase and beta-galactosidase; after de-glycosylation, only the GalNAc sugar moiety remains attached to Thr420, and it may interact with target proteins that have a string of complementary acidic amino acids.

Vitamin D physiology has gained more importance and publicity than any other component of the vitamin D axis as well as of its counterparts in water- and fat-soluble vitamin groups combined. This is partly because vitamin D deficiency is still widely prevalent both in the industrialized and in the developed world, and because it was demonstrated that the beneficial effects of vitamin D extend beyond the regulation of calcium and phosphorus homeostasis alone (Endocrinol Metab Clin North Am, 2010; 39:355-63). Vitamin D₃, known for centuries to affect mineral homeostasis, has several other diverse physiologic functions including effects on growth of cancer cells and protection against certain immune disorders (Endocrinol Metab Clin North Am, 2010; 39:243-53). Numerous studies revealed the protective effect of vitamin D against cancers, intermediate markers of cardiovascular risk, epidemic influenza, albuminuria, risk of fall and HIV infection (Rev Med Liege. 2013 January; 68(1):25-31). Further activities of vitamin D relate to defense of microbial infections, e.g. tuberculosis, contractility of muscle cells and counteraction of congestive heart failure. Cross-sectional studies demonstrated that vitamin D deficiency in humans is associated with elevated blood pressure and progression of atherogenesis, and vitamin D supplementation in adults may be regarded as simple means with few potential side effects to prevent atherogenesis or halt its progression and combat arterial hypertension (Int Urol Nephrol, 2010; 42:165-71).

It is immediately evident how these features of vitamin D are related to the progression of a number of chronic diseases with particular reference to those diseases that are associated with aging of the population, cardiovascular diseases and cancer just to name the most common. It is also evident how vitamin D supplementation is an almost mandatory requirement for practically all chronic disease. In fact, emerging evidence suggests that the progression of chronic diseases and many of the cardiovascular complications associated with them are linked to hypovitaminosis D. As new evidence has improved the understanding of classical (genomic), as well as the non-classical (rapid non-genomic), functions for vitamin D, it has become apparent that vitamin D acting as a secosteroid hormone is an important modulator of several systems including the immune, renal, nervous and cardiovascular systems (Ethn Dis, 2009; 19:S5-8-11). In particular, vitamin D produced in the kidney is known to have classical endocrine phosphocalcic properties as well as autocrine and paracrine actions on cellular proliferation and differentiation, apoptosis, renin secretion, interleukin and bactericidal proteins production (Med Sci (Paris), 2010; 26:417-21). It is evident that the effects on cell proliferation, differentiation and apoptosis are strictly connected with the anti-cancer properties of the vitamin D. In addition, epidemiological studies in chronic kidney disease, an increasing occurrence in modern societies, demonstrated that vitamin D deficiency and absence of treatment with vitamin D is associated with increased cardiovascular mortality (Ugeskr Laeger, 2009; 171:3684-9). Therefore, it is not surprising that clinical studies have consistently shown that vitamin D supplementation in patients with its deficiency, contribute to decreased frequency of bone disorders, cardiovascular incidents, lower risk of several malignancies and to improvement of immune system response regardless of renal function (Pol Merkur Lekarski, 2009; 27:437-41). The concept that vitamin D could represent a “panacea” was recently stressed in a peer-reviewed publication jokingly entitled “Does vitamin D make the world go ‘round’?” (Breastfeed. Med. 2008, 3(4), 239-250).

Several possible mechanisms may explain why vitamin D is so important in maintaining health. A number of studies indicate vitamin D exerts anti-inflammatory and immunomodulatory effects, thus counteracting the basic pathologic alterations that underlay all chronic conditions independently of their aetiology (Nat Rev Nephrol, 2009; 5:691-700).

The concept of “immunomodulation”, leads to the intricate relationship between vitamin D and the immune system, a system frequently compromised and/or deranged in all chronic conditions, from cancer to HIV infection. In fact, vitamin D stimulates the innate immune system, facilitating the clearance of infections such as tuberculosis and HIV.

Consistent with this concept, hypovitaminosis D has been associated with several autoimmune disorders, various malignancies, and cardiovascular risk factors in a number of recent epidemiological reports. Based on these observational reports, vitamin D and its analogues are being evaluated for the prevention and treatment of a variety of conditions (Curr Opin Nephrol Hypertens, 2008; 17:408-15). The net effects of vitamin Don the immune system, however, cannot be simply defined as immunostimulant. In fact, in some instances, vitamin D acts as an immunosuppressant and this might explain the observed beneficial effects in autoimmune disorders (Expert Rev Respir Med. 2012 December; 6(6):683-704).

For example, vitamin D induces the transcription of “endogenous antibiotics” such as cathelicidin and defensins and it inhibits the genesis of both Th1- and Th2-cell mediated diseases. It reduces the prevalence of asthma. Th1-dependent autoimmune diseases (e.g., multiple sclerosis, Type 1 diabetes, Crohn's disease, rheumatoid arthritis and so on) are also inhibited by vitamin D due to inhibition of antigen presentation, reduced polarization of Th0 cells to Th1 cells and reduced production of cytokines from the latter cells. In addition, supplementation of vitamin D has proven useful in the prevention or adjunct treatment of chronic obstructive pulmonary disease. Because of the complexity of the actions of vitamin D on the immune system the term “immunomodulation” referred to vitamin D appears to be fully justified (Curr Opin Pharmacol, 2010; 10:482-96).

All the considerations quoted above lead to the trivial conclusion that in the modern world, where people are not sufficiently exposed to the sun and do not produce enough endogenous vitamin D₃, vitamin D supplementation is almost always required for the maintenance of health. Much less trivial is the answer to the logically consequent question: “How much vitamin D has to be supplemented?” As recently discussed in a major Journal devoted to kidney research (Kidney Int, 2009; 76:931-3), the answer to that question does not depend solely on vitamin D serostatus, but it has to take into consideration the functionality of the receptor that is in turn influenced by the individual polymorphisms of the gene coding for the classical VDR. It is known that also the other components of the vitamin D axis play a role in determining the adequacy of vitamin D supplementation, thus rendering the answer to that question even more complex. I have found that by focussing on the role of VDBP and VDR in mediating the effects of vitamin D; a novel family of vitamin D-based supplements may be developed.

Further data with respect to VDBP is available from the published scientific literature. In addition to the various citations noted above, reference should be made for further background material about VDBP to the following papers:

Biochemical and Biophysical Research Communications, Vol. 163, No. 1, 1989, Calvo M, et al, “Relations between vitamin D and fatty acid binding properties of vitamin D-binding protein”, pages 14-17.

The Journal of Steroid Biochemistry and Molecular Biology, Vol. 42, No 8, September 1992, Bouillon, R, et al., “Polyunsaturated fatty acids decrease the apparent affinity of vitamin D metabolites for human vitamin D-binding protein”, pages 855-861.

Trends in Biotechnology, Vol. 2, No 7, No 7, July 2004, Gomme, P, et al., “Therapeutic potential of vitamin D-binding protein”, pages 340-345.

Nutrients, Vol. 5, No 7, July 2013, Thyer, L, et al., “A novel role for a major component of the vitamin D axis: vitamin D binding protein-derived macrophage activating factor induces human breast cancer cell apoptosis through stimulation of macrophages”, pages 2577-2589.

Proceedings of the Society for Experimental Biology and Medicine, Vol. 212, No 4, September 1996, Ray, R, “Molecular recognition in vitamin D-binding protein”, pages 305-312.

VDBP and the Modulation of Vitamin D Function

Vitamin D₃ is a hydrophobic molecule and as such it is not soluble in biological hydrophilic fluids. Therefore, it is physiologically bound to a specific binding protein that carries it from the blood to target cells in proximity of the cellular plasma membrane that is an hydrophobic structure. The protein that specifically binds vitamin D₃ and carries it to the plasma membrane of target cells is VDBP. Once at the plasma membrane, vitamin D₃ interacts with and activates the VDR. Activated VDR interacts with a number of intracellular signalling proteins and eventually leads to all the effects of vitamin D described in the previous paragraph (Steroids. 2001 March-May; 66(3-5):213-21). It can be stated that all the biological effects of vitamin D₃ are mediated by its interaction with VDBP and VDR.

VDBP is a serum alpha 2 glycoprotein composed of a single polypeptide chain with a molecular mass of 51-58 kDa and is structurally related to serum albumin. It is also known as Gc-globulin (Group-specific component globulin), is synthesized in the liver and is present in plasma at levels of 20-55 mg/100 ml. VDBP has been detected on the surface of several cell types, yolk sac endodermal cells, and some T lymphocytes. In B cells, VDBP participates in the linkage of surface immunoglobulins. The protein is 458 residues in length (J Biol Chem, 1986; 261:3441-50), and forms three domains, the first of which contains the vitamin D binding site. The three domains share limited sequence homology with each other and with similar repeats in human serum albumin.

In addition to these well-known homologies, we have observed, however, that there are sequence homologies with the other component of the vitamin D axis, that is the VDR. FIG. 1 illustrates the amino acid sequence of the three isoforms of VDBP and the alignment with the amino acid sequence of VDR. The tract between amino acid 1 and 197 (exons 1, 2, 3, 4, 5) shows 20% identity between VDBP and VDR; the tract between amino acid 217 and 330 (exons 6, 7, 8) shows 40% identity. These characteristics led to the hypothesis that VDBP and VDR might interact with each other, thus mediating the effects of vitamin D₃ as explained in detail in the following paragraphs.

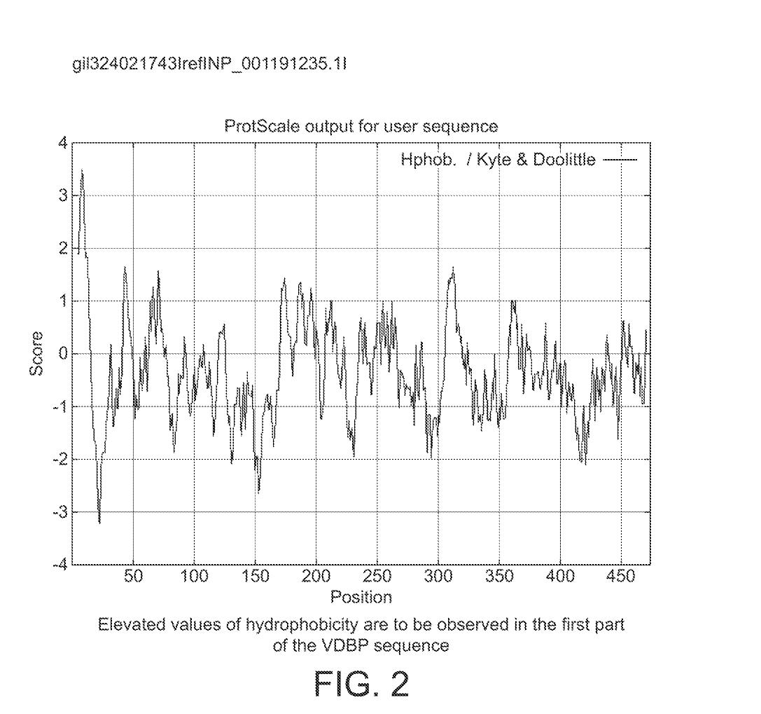

A more detailed analysis of the hydrophobic profile of VDBP is shown in FIG. 2 where the method of Kyte and Doolittle is used to calculate the relative hydrophobicity of the amino acid sequence. It can be observed that the first part (amino terminus) of the VDBP sequence shows high values of hydrophobicity that correspond to the region where vitamins D₃ and/or its analogues bind through hydrophobic interactions.

The key difference between what has been done in the past and the design of the novel family of molecules in accordance with the present invention is that instead of aligning the amino acid sequences of VDBP and VDR as it is classically done starting from the amino terminus and as shown in FIG. 1, the alignment is reversed and I compared the amino acid sequence of the amino terminus of VDBP, where vitamin D binds, is compared with the carboxyl terminus of VDR that is the hydrophobic region where vitamin D binds to VDR. In other words, the two hydrophobic sequences of VDBP and VDR that are the regions where vitamin D binds to both molecules are aligned. In order to do this, the sense of the alignment, aligning the first part of VDBP with the last part of VDR has to be reversed, as if the two sequences were anti-parallel. This novel type of reasoning was inspired by the alignment of the nucleotide sequences in DNA where the sense of the two strands is anti-parallel.

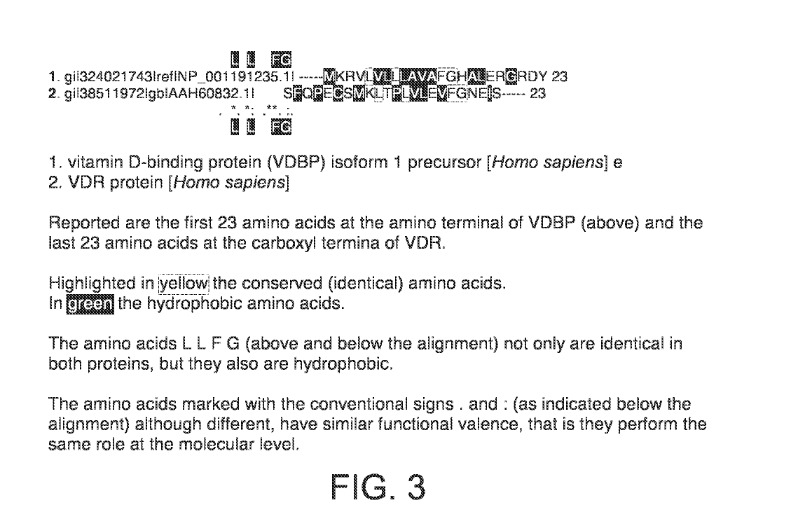

FIG. 3 shows the alignment of the first 23 amino acids of VDBP with the last 23 amino acids of VDR. These are the regions where vitamin D and/or its analogues bind.

It can be observed that there are 23 hydrophobic amino acids near the amino terminus of VDBP (-----MKRVLVLLLAVAFGHALERGRDY) and 23 amino acids near the carboxyl terminus of the VDR (SFQPECSMKLTPLVLEVFGNEIS-----). If these two sequences are aligned as shown in FIG. 3, it is possible to observe not only that in both proteins there is a long stretch (13-14) of hydrophobic amino acids (highlighted in green), but that 4 hydrophobic amino acids are identical (L L FG; indicated in yellow and in green above and under the alignment. The sequence of VDBP is above), and 11 amino acids have similar functional valence (as indicated by the conventional symbols [*], [.] and [:]. Therefore, in the vitamin D binding domains of VDBP and VDR there are in total 11 out of 23 amino acids that show functional identity or similarity, and 13-14 that are hydrophobic.

The novel family of compounds according to the invention is thus based on the discovery that VDBP and VDR interact through their conserved vitamin D-binding hydrophobic domains, and that vitamin D could be sandwiched between the two proteins.

In addition to this type of hydrophobic interaction, we have found that when VDBP is selectively de-glycosylated at Thr 420, another site of molecular interaction with VDR, this time of the hydrophilic type, can be exposed.

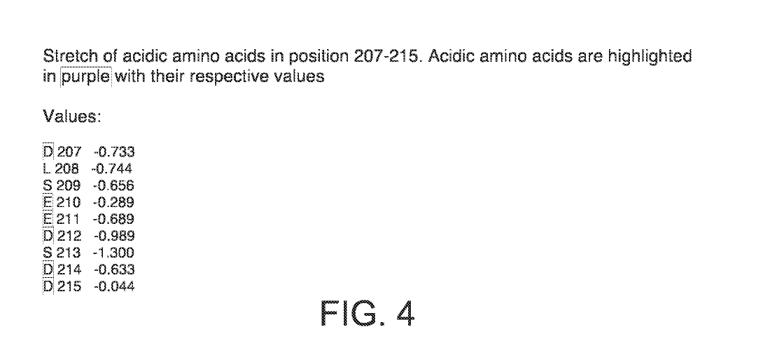

In fact, as stated in the previous paragraph, naturally occurring VDBP isoforms termed Gc1s and Gc1f carry a linear O-linked trisaccharide of the type GalNAc-Gal-Sia that is attached to Thr 420 (Biochim Biophys Acta. 2010 April; 1804(4):909-17). The use of enzymes such as sialidase and beta-galactosidase leads to the exposure of the alpha-N-acetylgalactosamine (GalNAc) moiety that has hydrophilic, basic, chemical-physical characteristics. Therefore, GalNAc at position Thr 420 can interact with the stretch of acidic amino acids that is located in position 207-215 of the VDR. In this position there are 6 amino acids in close proximity that identify an acidic pouch that binds GalNAc (FIG. 4).

Therefore, thus far, two sites of interaction between VDBP and VDR have been identified: 1. The hydrophobic stretch of 23 amino acids where vitamin 03 binds to both molecules; 2. The hydrophilic stretch of acidic amino acids in VDR where the GalNAc moiety of de-VDBP binds.

In addition to the two binding sites described above, we have discovered another site of interaction between VDBP and VDR, this time in a region of VDBP that is not involved in vitamin 03 binding.

In fact, in the region located between domains II and III, that is between the positions 304 and 387, VDBP shows a shallow cleft that binds fatty acids (Biochem Biophys Res Commun. 1988 Jun. 30; 153(3):1019-24); the fatty acids located in this shallow cleft can interact with the cellular plasma membrane thus favouring the interaction between the VDBP on the outer side of the membrane and the VDR located inside the cell. When I aligned the 23 hydrophobic amino acids of the VDR quoted above (i.e. those that bind vitamin D₃) are aligned with the corresponding hydrophobic amino acids of the unsaturated fatty acid binding site of VDBP (in particular, those in position 356-386), a significant degree of functional homology can be observed: in fact there are 8 amino acids with similar functional valence in a long stretch of hydrophobic amino acids.

The observation that VDBP has multiple binding/recognition sites is not surprising. Thus, it is well known that VDBP is a multifunctional protein that, in addition to vitamin D, binds immunoglobulins and actin and even acts as an actin scavenger. The affinity for actin monomers is high and the actin binding site has been reported to reside within domain III, between residues 350 and 403 (see FIG. 1). The structure of the complex of VDBP and actin (Proc Natl Acad Sci USA, 2002; 99:8003-8) confirms that domain III forms an actin-binding contact between sub-domains 1 and 3 of actin. These characteristics of VDBP, even though not directly related to vitamin D metabolism, are of paramount importance in chronic conditions that involve cell necrosis such as cancer and cardiovascular diseases, since actin is the most abundant protein in eukaryotic cells and is a major cellular protein released during cell necrosis that may cause fatal formation of actin-containing thrombi in the circulation if the actin scavenging capacity of VDBP is exceeded (Dan Med Bull, 2008; 55:131-46). Thus, recent studies demonstrated that determination of serum level of VDBP is useful as a prognostic indicator in patients with acute hepatic failure, acetaminophen overdose, multiple trauma or multiple organ dysfunction syndrome, or sepsis. Other studies suggest an association between VDBP levels and resistance or susceptibility to chronic obstructive pulmonary disease, thyroid diseases, diabetes, multiple sclerosis, and sarcoidosis (Postepy Hig Med Dosw, 2008; 62:625-31).

In addition it should be noted that VDBP does not bind uniquely vitamin D₃ at the 23 hydrophobic amino acid binding domain. Other compounds, termed vitamin D analogues and able to bind and activate the VDR, show affinity for VDBP although generally lower than that of vitamin D₃. For example, two non-hypercalcemic vitamin D analogues, calcipotriene and 22-oxacalcitriol show low VDBP affinity that has been held responsible for the reduced calcemic actions (Am J Kidney Dis, 1998; 32:S25-39).

VDBP and VDR Signalling

Until recently, the signalling mechanism of vitamin D₃ was described as follows: vitamin D₃ a highly hydrophobic molecule, is carried in blood and biological fluid by VDBP. Once at the level of the cellular plasma membrane (a highly hydrophobic structure), vitamin D₃ is released, it freely crosses the plasma membrane, and, once inside the cell, interacts with its proteinic receptor that is the VDR. The dimeric complex vitamin D₃/VDR translocates to the nucleus where it interacts with a number of signalling proteins and controls the expression of a number of different genes that are ultimately responsible for the biological effects of vitamin D₃. Since the VDR is expressed in a huge number of normal and pathologic tissues, this explains the numerous and multifaceted effects of vitamin D₃ in physiology and pathology (for rev, see: Proc. Natl. Acad. Sci. U.S.A. 101 (16): 6062-7). In this scenario, the role of VDBP were simply to carry vitamin D₃ in blood and in biological fluids and not to participate in the signalling mechanism.

However, more recent studies demonstrated that this scenario is incomplete. In fact, it was demonstrated that vitamin D₃ does not cross freely the plasma membrane, but that the complex VDBP/vitamin D₃ is internalized into the cells and, once inside the cell, it binds to the VDR. Internalization is performed by plasma membrane-associated proteins called megalin, cubilin and the adaptor protein disabled-2 (J Nutr. 2008 July; 138(7):1323-8). According to this scenario, VDBP is essential for the interaction between vitamin D₃ and VDR.

However, as described in the preceding section. VDBP can exist in two forms, fully glycosylated and de-glycosylated. According to the model proposed in the preceding section, the de-glycosylated form of VDBP can establish a more stable interaction with VDR thanks to the hydrophilic interaction between the GalNAc at Thr 420 and the string of acidic amino acids of VDR described above.

This scenario might appear odd at first, since for many years it had been thought that VDR was localized in the cytoplasm and in the nucleus, whereas VDBP could not cross the plasma membrane and therefore had to be recognized by a surface receptor, possibly a lectin-type receptor (J Biol Chem, 1999; 274:10697-705). However, the observation of an association between the polymorphisms of the gene coding for VDR and differential responses to de-VDBP in human monocytes (J Nephrol. 2012 July-August; 25(4):577-81) as well as with metastatic breast cancer (Oncol Res. 1998; 10(1):43-6), support the apparently odd issue of a direct molecular interaction between de-VDBP and the VDR. In support for this hypothesis there is the observation that the VDR translocates to the plasma membrane (J Cell Biochem. 2002; 86(1):128-35), and plasma-membrane associated VDR is responsible for the rapid, non-genomic effects of vitamin D (Calcif Tissue Int (2013) 92:151-162).

According to this novel signalling scenario, the signal transduction mechanism of vitamin D₃ is the following: VDBP carries vitamin D₃ and a fatty acid as described above, thus forming a trimeric complex. This complex interacts with the plasma membrane through the hydrophobic portions of VDBP where vitamin D₃ and the fatty acid are bound. The complex is internalized by cellular proteins. Once inside the cell, the complex interacts with membrane-associated VDR. If VDBP is fully glycosylated, the interaction is mediated only through hydrophobic interactions; however, if VDBP is de-glycosylated and GalNAc exposed, the interaction with VDR is more stable since it involves hydrophilic, base-acid interactions. In this latter case, the tetrameric complex that is [vitamin D₃/de-VDBP/fatty acid/VDR] translocates to the nucleus where it interacts with other signalling proteins and with DNA, thus regulating the multitude of genes that are known to be modified by activated VDR.

In summary; it is the complex [vitamin D₃/VDBP/fatty acid] that activates VDR and is responsible for the effects so far attributed to vitamin D₃. The de-glycosylated form of VDBP is more efficient in activating VDR because it can establish a more stable interaction.

The object of the present invention is thus to provide an entire family of novel molecules/complexes able to activate VDR in a more efficient way, using the de-VDBP as backbone.

Based on what has been described above, this family of molecules/complexes should desirably have the following advantages in comparison with the current supplements made of vitamin D that are being used in the variety of conditions described in section 4.1 above.

1. A much greater potency since they will mimic the actual molecular arrangement that occurs in nature.

2. A much greater stability and therefore a greater efficiency since they will use de-VDBP as backbone.

3. The approach should enable a great variety of new molecules to be designed by substituting vitamin D₃ and fatty acids on the de-VDBP backbone with other VDR agonists and/or fatty acids endowed with properties that are known to be beneficial for specific health conditions. Each type of new molecule/complex could then be targeted to a particular disease or condition.

4. Tailor-made molecules/complexes that take into account the genetic polymorphism of the VDR of each individual subject can be designed.

5. These novel molecules/complexes can be encapsulated into liposomes; this cannot be done with regular vitamin D₃. Liposomes can be consumed orally and deliver their content in plasma with an efficiency that compares favourably with that obtainable using intravenous injection.

### GENERAL DESCRIPTION OF THE INVENTION

The present invention provides molecules or multimolecular complexes based on the backbone of de-VDBP, to which vitamin D (or its analogues) and fatty acids are bound via hydrophobic interactions. This novel family of molecules/complexes will provide all the known beneficial effects of vitamin D₃ supplementation with the advantage that these new molecules and new complexes of molecules, will be more stable and more active, can be specifically designed to target specific diseases and/or to meet individual genetic variables, and can be encapsulated in liposomes for efficient delivery.

In fact, up until now, vitamin D₃ is provided as a supplement for oral or parenteral administration, and, in the preparations currently in commerce, Vitamin D₃ is not complexed with any other molecule; in particular, in no preparation it is complexed with its naturally occurring binding protein that is VDBP, let alone with de-VDBP. Therefore, when vitamin D₃ is ingested (or administered), it binds to plasma VDBP and it is carried to the cells where it exerts its actions as described in section 4.1 above. However, since it is not complexed with de-VDBP, the interaction with its receptor, VDR, is not as stable and efficient as it could be if it were complexed with de-VDBP as proposed in the present invention.

According generally to the present invention, there is provided a process for the production of modified vitamin D-containing complexes which includes the following steps:

1. VDBP is sequentially de-glycosylated e.g. according to the method described in (Biochim Biophys Acta. 2010 April; 1804(4):909-17).

2. Vitamin D₃ or its analogues are bound to de-VDBP through hydrophobic interactions.

3. Unsaturated fatty acids (UFA) are bound to the complex [vitamin D₃/de-VDBP].

These trimeric complexes [vitamin D₃/de-VDBP/UFA] may be encapsulated in liposomes to enable oral administration or topical administration, for example in an ointment. Alternatively, the complexes may be used as such in a suitable carrier for sub-lingual administration. In a further alternative, they may be used in mixtures suited for intra- or perk tumoral injection.

By carrying out this process, a family of new molecules and multimolecular complexes where vitamin D (or its analogues) and fatty acids are bound to de-VDBP may be produced. The number of new molecules that can be produced with this approach is very high since vitamin D₃ can be substituted for with a variety of vitamin D analogues and several UFA can be made to interact with the specific binding site in the de-VDBP molecule.

The element of novelty in this invention lies in the original design of multimolecular complexes that mimic the natural molecular arrangement of vitamin D₃ as it occurs in physiological signalling. This design is based on our observations concerning the molecular structures of VDBP and VDR. The advantages of these molecules over the existing preparations are listed in the previous section. The usefulness of these molecules lies in the fact that they are more active, more stable and more specific than existing vitamin D supplements and can be substituted for existing vitamin D supplements in all their numerous applications.

### SPECIFIC DESCRIPTION OF PREFERRED EMBODIMENTS

A General Method for the Preparation of New Molecules or Mixtures of Molecules According to the Present Invention is Set Out in Section 6.1.1 to 6.1.5 Below:

De-Glycosylation of VDBP.

De-glycosylation of VDBP was achieved according to the method of Ravnsborg et al. (Biochim Biophys Acta. 2010 April; 1804(4):909-17) as modified in Bradstreet et al. (Autism Insights 2012:4 31-38). Briefly, VDBP was isolated from purified human serum obtained from the American Red Cross using 25-hydroxyvitamin D₃-Sepharose high affinity chromatography or actin-agarose affinity chromatography. The bound material was eluted and then further processed by incubation with three immobilized enzymes. The resulting de-VDBP was filter sterilized. The protein content and concentration was assayed using standard Bradford protein assay methods (Anal. Biochem. 1976; 7: 248-254).

Binding of Vitamin D₃ to De-VDBP and Preparation of the First Members of the New Family of Multimolecular Complexes.

Vitamin D₃ [1α,25-Dihydroxyvitamin D₃ (6,19,19-d3)] was obtained from Sigma-Aldrich. Its molecular weight is 419.61 Da by atom % calculation. Incubation with de-VDBP was performed in a test tube at 25° C. for 30 min gently shaking. The ratio vitamin D₃/de-VDBP was calculated considering the molecular weight of de-VDBP as 58 kDa. The following ratios were used: 1/10; 2/10; 5/0; 1/1 (where the first number refers to the calculated number of vitamin D₃ molecules and the second number refers to the calculated number of de-VDBP molecules. Thus, the ratio 1/1 indicates that there was one molecule of vitamin D₃ per each molecule of de-VDBP). In order to favour the hydrophobic interaction between vitamin D₃ and de-VDBP, incubation buffers with different ionic strength were used, according to the principles outlined in Cecchi et al. (Clin Chim Acta. 2007 February; 376(1-2):142-9). Essentially, the ionic strength was increased when the ratio vitamin D₃/de-VDBP increased. In this manner, the hydrophobic interaction between a higher number of molecules was favoured and stabilised. The concentration of NaCl in the incubation buffer ranged from 0.2 to 2.0 M. In other experiments, guanidine hydrochloride instead of NaCl was used according to the methods described in Ital J Anat Embryol. 2001 January-March; 106(1):35-46. After incubation, the samples were exhaustively (24 h) dialyzed against a dialysis membrane with cut-off set at 60 kDa in order to remove excess salt. After 24 h dialysis, the NaCl concentration was adjusted to that of the so called physiological saline solution. The final concentration was adjusted taking into account de-VDBP. Exempli gratia, in a vial with a concentration of 100 ng/ml, this refers to the concentration of de-VDBP, independently of whether there were other molecules (vitamin D₃ or UFA) bound to it, see below.

At the end of the procedure, a series of new molecules and mixtures of new molecules was obtained depending on the ratio vitamin D₃/de-VDBP that was used. To simplify, when the ratio was set at 1/10, in the mixture there were 10 molecules of de-VDBP and only 1 molecule of vitamin D₃. Therefore, only 1 molecule of de-VDBP was complexed (i.e. associated) with 1 molecule of vitamin D₃. The remaining 9 molecules of de-VDBP, because of the ionic strength in the incubation medium, interacted with each other through their respective hydrophobic domains. However, since the number of de-VDBP was odd (i.e. it was 9), 1 molecule of de-VDBP remained “free”. Thus, in extremely simple terms, it can be stated that in the ratio 1/10 there were three types of molecules: 1 molecule of de-VDBP complexed with vitamin D₃ (i.e. the dimeric complex [vitamin D₃/de-VDBP]; 1 molecule of de-VDBP free to interact with its receptor or other molecules; 4 pairs of de-VDBP where in each pair one de-VDBP molecule was complexed with another de-VDBP molecule. Each one of these three types of molecule could then perform a distinct action at the cellular level and, consequently, on the entire organism. The dimeric complex [vitamin D₃/de-VDBP] binds to the cellular plasma membrane, interacts and activates the VDR and triggers the signalling cascade described in the “background” section (4.3). The “free” de-VDBP binds to its chondroitin sulfate proteoglycan receptor on the extracellular part of the plasma membrane triggering its signalling cascade (J Immunol. 1999 Aug. 15; 163(4):2135-42). Each one of the four pairs of de-VDBP is internalised into the target cells through cellular transport proteins as described in (Mol Immunol. 2007 March; 44(9):2370-7).

The activity of molecules produced with this method can be further modulated by dissolving them in an aqueous alcoholic saline solvent. This particular type of excipient is routinely used in intravenous preparations. We have found that improved results can be secured using a mixture of equal parts of saline and an aqueous mixed alcohol solvent (20% v/v ethanol, 30% v/v) propylene glycol, balance water. Since this excipient is partially hydrophobic, the interaction between de-VDBP in the pairs described above can be disrupted. Therefore, in its presence, there will be one dimeric complex [vitamin D₃/de-VDBP] and 9 “free” de-VDBP molecules. Quite obviously, this latter combination will perform different actions at the cellular and organism level. For example, this combination could be used in advanced cancer where extensive necrosis and release of actin from necrotic cells occurs; in this case, the 9 “free” molecules of de-VDBP could act as actin scavengers and remove toxic actin, whereas the dimeric complex [vitamin D₃/de-VDBP] could stimulate cell responses as noted in the “background” section above.

Such a solvent can also be used to solubilise de-VDBP prepared as described in 6.1.1. In fact, in a saline solution where there are only de-VDBP molecules, the ionic strength will cause most of them to interact with each other through their respective hydrophobic domains and only a minority of them will remain “free” to interact with its receptor or other molecules. It can be stated that because of these interactions, the biological activity of de-VDBP in saline is very low. It can be increased by orders of magnitude by solubilising it in an aqueous alcoholic saline solvent.

Binding of Unsaturated Fatty Acids to the Dimeric Complex [Vitamin D₃/De-VDBP].

Unsaturated fatty acids were obtained from Sigma-Aldrich. They were:

i. Oleic acid, a monounsaturated omega-9 fatty acid, abbreviated with a lipid number of 18:1 cis-9 with the formula

CH₃(CH₂)7CH═CH(CH₂)7COOH.

ii. Eicosapentaenoic acid (EPA or also icosapentaenoic acid), an omega-3 fatty acid. In chemical literature, it is given the name 20:5(n−3). EPA is a carboxylic acid with a 20-carbon chain and five cis double bonds; the first double bond is located at the third carbon from the omega end.

Both UFA were chosen because of four characteristics: the high hydrophobicity; the ability to bind to the shallow cleft of hydrophobic amino acids in de-VDBP; the flexibility inherent in their molecular structure due to presence of double bonds in cis configuration; their well-documented health-promoting properties.

Other poly-UFA were also considered as described below.

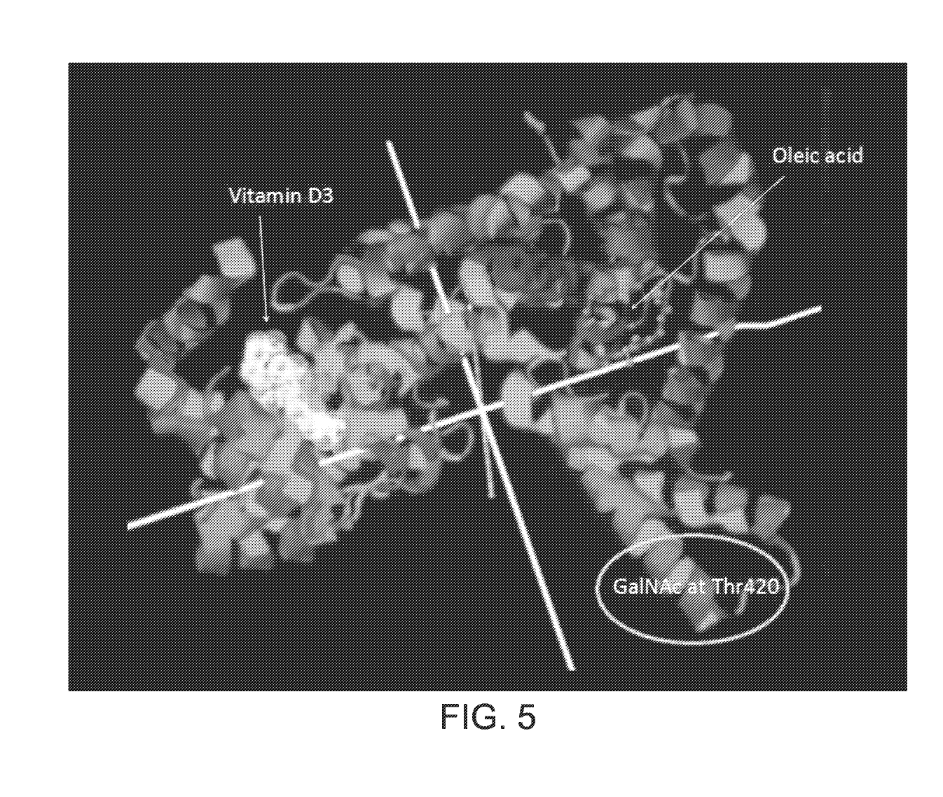

UFA were incubated with dimeric [vitamin D₃/de-VDBP] complexes following the procedure described in the preceding paragraph. The ratio vitamin D₃/de-VDBP]/UFA was calculated taking into account the number of de-VDBP molecules in each [vitamin D₃/de-VDBP] complex. For example, in the composition resulting from a ratio 1/10 vitamin D₃/de-VDBP, there were 1 molecule of [vitamin D₃/de-VDBP] and 9 molecules of de-VDBP (bound in pairs or free). When UFA (e.g. oleic acid) was added at a ratio 1/1, this means that there were as many molecules of de-VDBP as oleic acid; therefore, in the final mixture there were: 1 molecule of the trimeric complex [vitamin D₃/de-VDBP/oleic acid]; 9 molecules of the dimeric complex [de-VDBP/oleic acid]. Instead, when oleic acid was added at a ratio 1/10, this means that in the final mixture there was only 1 trimeric complex [vitamin D₃/de-VDBP/oleic acid]; and 9 de-VDBP molecules that were bound in pairs or free. A model rendering of a trimeric complex [vitamin D₃/de-VDBP/oleic acid] is depicted in FIG. 5. Both vitamin D₃ and oleic acid are clearly visible bound to their respective hydrophobic binding sites located in shallow clefts of the protein (arrows); the occurred de-glycosylation of VDBP is manifested by the presence of GalNAc at Thr 420 as indicated in the oval.

Using this procedure a high number of possible combinations could be obtained in a rapid and simple way taking into account 4 variables: i. the ratio vitamin D₃/de-VDBP; ii. the ratio [vitamin D₃/de-VDBP]/UFA; iii. the type of UFA; iv. the presence of the aqueous alcoholic saline solvent.

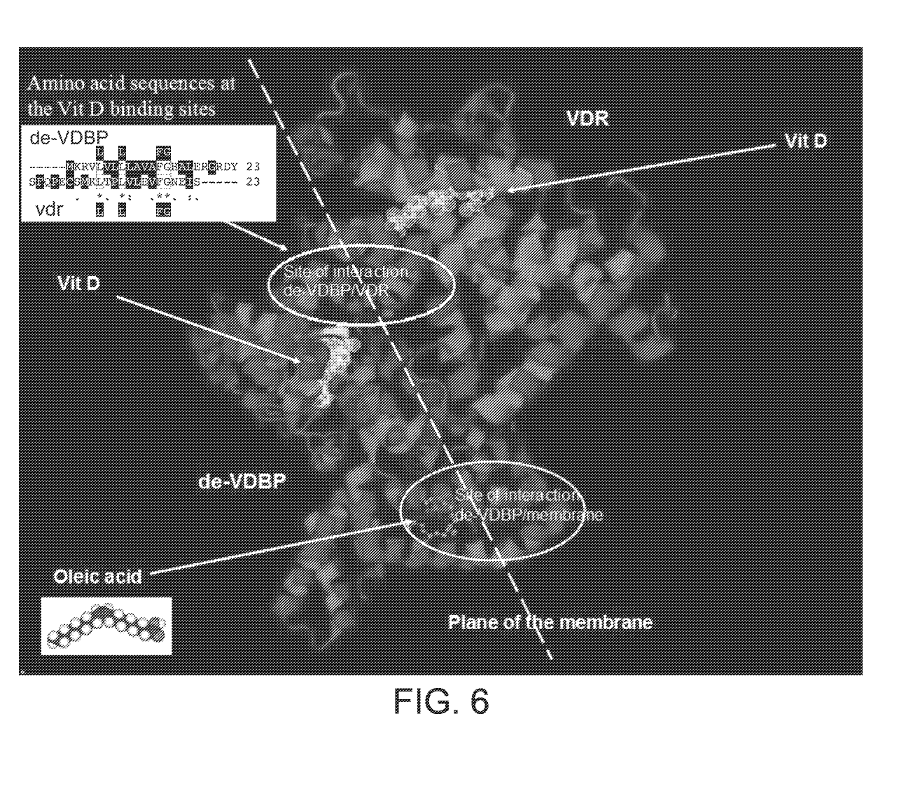

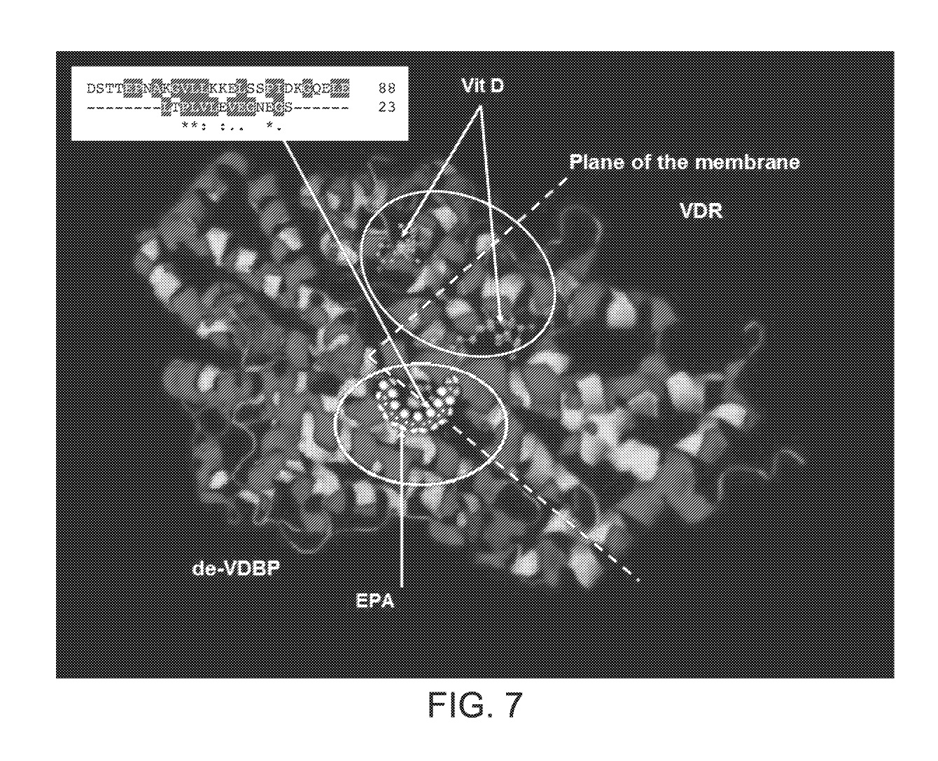

The combinations thus obtained can be targeted to specific conditions where the use of vitamin D₃ has already been demonstrated beneficial. For example, a combination where the majority of the molecules belongs to the trimeric type [vitamin D₃/de-VDBP/oleic acid] could be used for maximal stimulation of VDR signalling in conditions where the lipid composition of the plasma membrane is normal. In fact, as demonstrated in the molecular rendering presented in FIG. 6, the presence of oleic acid, a rather “straight” molecule, confers a somewhat linear interaction between the two recognition domains mediating de-VDBP/VDR interaction that are: i. the vitamin D₃ binding domain; ii. the GalNAc/acidic amino acid recognition domain. However, when the trimeric complex is [vitamin D₃/de-VDBP/EPA]as reported in FIG. 7, since EPA is a poly-unsaturated fatty acid with a more “curves” configuration, interaction of EPA with the plasma membrane determines a different spatial interaction between the complex [vitamin D₃/de-VDBP/EPA] and VDR. In the box in the left upper corner of the figure the complementary hydrophobic amino acid sequences of de-VDBP and VDR are reported. The upper line refers to the de-VDBP sequence where EPA is bound; the lower line refers to the hydrophobic 23 amino acid sequence of VDR. It is possible to notice that that there is a significant degree of functional homology where EPA is bound; in fact there are 8 amino acids with similar functional valence in a long stretch of hydrophobic amino acids (highlighted in blue in FIG. 7). The overall configuration of the tetrameric complex [vitamin D₃/de-VDBP/EPA/VDR] described in FIG. 7 is more globular than the relatively linear tetrameric complex [vitamin D₃/de-VDBP/oleic acid/VDR] depicted in the FIG. 6. Therefore, as it will be described in the following sections, a trimeric complex [vitamin D₃/de-VDBP/EPA] could be more efficient in all those diseases where EPA has been demonstrated to be beneficial, from chronic inflammation to cancer and neurological diseases. In fact, The US National Institute of Health's MedlinePlus lists medical conditions for which EPA (alone or in concert with other w-3 sources) is known or thought to be an effective treatment and most of these involve its ability to lower inflammation. A trimeric complex such as the one described above would combine the well demonstrated beneficial properties of vitamin D₃ and EPA in a single, stable, and signalling-efficient molecule.

Substitution of Vitamin D₃ with Analogues.

In the dimeric or trimeric complexes described above, vitamin D₃ can be substituted for with different analogues that are able to stimulated the VDR and trigger the signalling cascade described above, but that have different affinity for de-VDBP or VDR. Vitamin D analogues are molecules synthesized starting from the basic chemical structure of vitamin D₃ but with different affinity for VDBP and/or VDR (J Med Chem. 2012 Oct. 25; 55(20):8642-56). Most of them are synthesized in order to obtain compounds that have anti-proliferative, pro-differentiating, and transcriptional activities similar to those of native vitamin D₃, but with less pronounced hypercalcemic effects. Currently, their main field of application is in chronic kidney disease (Curr Vasc Pharmacol. 2013 May 16), cancer (Anticancer Agents Med Chem. 2013 Jan. 1; 13(1):118-25), diabetic nephropathy (Scientific World Journal. 2013 Apr. 24; 2013:928197), psoriasis (J Drugs Dermatol. 2013 May 1; 12(5):546-50) and prevention of fibrosis (Lab Invest. 2012 December; 92(12):1666-9). Some of the analogues that have been tested are: doxercalciferol, alphacalcidol, paricalcitol (19-nor-1,25-dihydroxyvitamin D₂), maxacalcitol (1,25-dihydroxy-22-oxa-vitamin D₃), calcipotriol (calcipotriene), and 22-oxacalcitriol (OCT). Most of these compounds have lower affinity for VDBP (Am J Kidney Dis, 1998; 32:S25-39). The procedure to prepare the dimeric and trimeric complexes was identical to that described in 6.1.2 and 6.1.3. Also in the case of these complexes, the aqueous alcoholic solvent can be included. These dimeric and trimeric complexes where vitamin D₃ is substituted for by one of its analogues, each of which has peculiar characteristics, can be indicated for all the conditions that are indicated for the analogues when they administered alone. Also in this case, the novelty of this invention lays in the use of de-VDBP as backbone to favour stabilisation of the analogue, interaction with a UFA and eventually more efficient signal transduction via the activated VDR.

Preparation of Liposomes Containing the Complexes Described Above.

A liposome is an artificially-prepared vesicle composed of a lipid bilayer. Liposomes can be used as a vehicle for administration of nutrients and pharmaceutical drugs. Liposomes can be administered orally and, once absorbed, they deliver their content in blood with an efficiency that is only slightly lower than that of intravenous injection. Because of these characteristics, encapsulation of hydrophobic and hydrophilic nutrients and pharmaceutical drugs within liposomes is a very effective method of bypassing the destructive elements of the gastric system and aiding the delivery of the encapsulated nutrient/drug to the cells and tissues. As of 2008, 11 drugs with liposomal delivery systems have been approved and six additional liposomal drugs were in clinical trials (Clin Pharmacol Ther. 2008 May; 83(5):761-9. Epub 2007 Oct. 24). These include:

Liposomal amphotericin B for fungal infections

Liposomal amphotericin B for fungal and protozoal infections

Liposomal cytarabine Depocyt for malignant lymphomatous meningitis

Liposomal daunorubicin DaunoXome for HIV-related Kaposi's sarcoma

Liposomal doxorubicin for combination therapy with cyclophosphamide in metastatic breast cancer

Liposomal IRIV vaccine for Hepatitis A

Liposomal IRIV vaccine for Influenza

Liposomal morphine for endo postsurgical analgesia

Liposomal verteporfin for age-related macular degeneration, pathologic myopia, ocular histoplasmosis

Liposome-PEG doxorubicin for HIV-related Kaposi's sarcoma, metastatic breast cancer, metastatic ovarian cancer

Micellular Estradiol

In preparing liposomes designed to encapsulate de-VDBP and the multimolecular complexes described in 6.1.1., 6.1.2., 6.1.3. and 6.1.4., we considered the molecular interactions between the GalNAc moiety of de-VDBP. In fact, all the other regions of the molecule and/or the complexes, do not present peculiar arrangements of electrical surface charges that need to be taken into account in designing specific liposomes.

The following lipid compositions (Sigma-Aldrich), all of them resulting in a negative surface charge, were considered:

1-palmitoyl-2-oleoyl-phosphatidylcholine (POPC):Cholesterol: 1-palmitoyl-2-oleoyl-phosphatidylglycerol (POPG) in a ratio of =30:40:30.

L-α-phosphatidylcholine, β-oleoyl-γ-palmitoyl, 8.7 μmol. L-α-phosphatidyl-DL-glycerol, dioleoyl, 1.0 μmol. cholesterol, 6.9 μmol.

L-α-phosphatidylcholine, distearoyl, 8.4 μmol. L-α-phosphatidyl-DL-glycerol, distearoyl, 0.9 μmol. cholesterol, 6.9 μmol

Liposomes were prepared using a LiposoFast Liposome Factory (Sigma-Aldrich). This methods allows for fast, efficient formation of uniform-sized, unilamellar liposomes. The principle is the following: a lipid emulsion is repeatedly extruded through a porous polycarbonate membrane, forced back and forth by specially modified gas-tight syringes attached to the membrane support capsule. With the syringes provided with the kit, unit has 0.5 mL capacity and virtually zero dead volume. Membranes from 200 to 400 nm pore size were used to produce liposomes of the desired size. Quite obviously, smaller size liposomes were used for single molecules such as de-VDBP; larger size liposomes were used for the di- and trimeric complexes described in sections 6.1.2, 6.1.3 and 6.1.4 above.

The novelty and the advantages of delivering all the novel compounds described above through liposomes is evident. Potent and stable molecules pertaining to the vitamin D axis can be administered orally and reach plasmatic concentration not dissimilar from that achievable through intravenous injection. Since liposomes are currently being used to efficiently deliver anti-cancer, anti-HIV and anti-microbial pharmaceutical agents, once used to encapsulate the novel multimolecular complexes described above, they can be used in all those conditions where vitamin D and its analogues have proven effective.

Preparation of Specific Molecules and Mixtures of Molecules Aimed at Specific Diseases or Condition According to the Methods Described in 6.1

General Principles.

The molecules and mixture of molecules that will be described below take into account the peculiar biomolecular characteristics of cells in different diseases and conditions and are designed according to the methods described in section 6.1. The general nomenclature of these preparation is the following: “I” stands for “injectable. “L” indicates the liposomal form that can be used orally or transdermally. “S” stands for saline and indicates that the compounds are dissolved in physiological saline solution. “X” stands for the aqueous alcoholic saline solvent specified in section 6.1.3 above. The letters of the Greek alphabet are used to indicate the names of the mixtures. The final de-VDBP concentration is 100 ng/ml. Other concentration can be easily achieved according to specific needs.

The following are descriptions of examples of molecules and mixtures of molecules in accordance with the present invention and intended as supplements or injectable preparations targeted to different types of cancer.

IX-Alpha/IS-Alpha. A mixture of molecules for advanced cancers, i.e. stage 4 and metastasized. This preparation takes into account the high level of toxic actin that is released from necrotic cancer cells. Therefore it is prepared starting with 1/10 ratio vitaminD₃/de-VDBP. Oleic acid at a ratio 1/10 is added. In this preparation there is the highest number of “free” de-VDBP molecules available to scavenge actin and to interact with the de-VDBP receptor. The trimeric complexes [vitamin D₃/de-VDBP/oleic acid] will interact with VDR at the plasma membrane and trigger the VDR signaling.

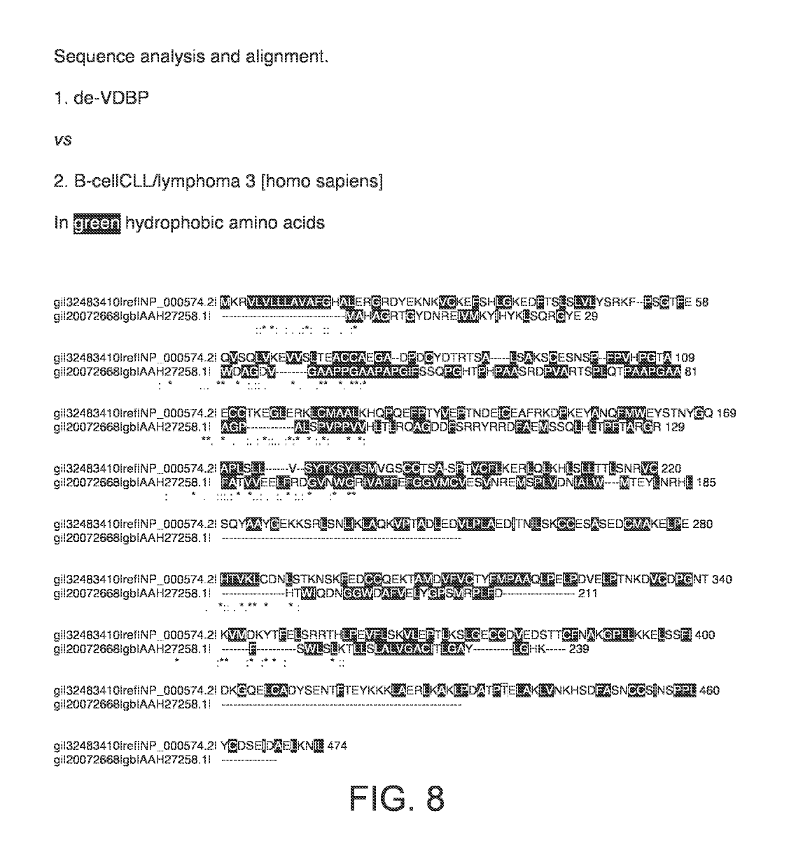

IS-Beta. A mixture of molecules for leukemias, lymphomas and other cancers where apoptosis is de-regulated. This preparation takes into account the hydrophobic profile of the gene Bcl-2, one the genes most frequently activated in these types of cancer. The comparison of the hydrophobic profile of de-VDBP and Bcl-2 is reported in FIG. 8. The preparation starts with 2/10 ratio vitaminD₃/de-VDBP in saline; oleic acid at a ratio 1/1 is added. In this preparation there is a high number of hydrophobic dimeric complexes [de-VDBP/oleic acid] that will interact through hydrophobic interactions with Bcl-2, thus inhibiting its action. In order to favor this interaction, the ionic strength of saline is used. The relatively few trimeric complexes [vitamin D₃/de-VDBP/oleic acid] will interact with VDR at the plasma membrane and trigger the VDR signaling.

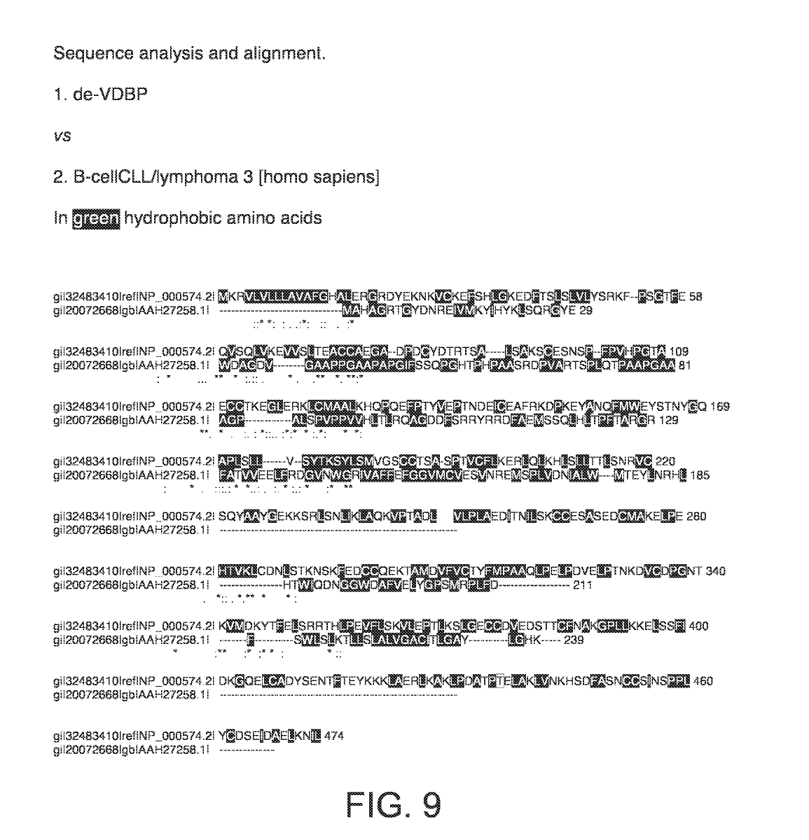

IX-Gamma/IS-Gamma. A mixture of molecules for colon and breast cancer. This mixtures of molecules targets the oncosuppressor gene product p53 that is involved in about 50% of human carcinomas (BMC Cancer. 2013 Jun. 5; 13(1):277). As it can be observed in FIG. 9, p53 shows a much less hydrophobic profile than Bcl-2. Therefore, the design of molecules targeted at this protein must take into account hydrophilic interactions involving the GalNAc moiety of de-VDBP. The preparation starts with 1/10 ratio vitaminD₃/de-VDBP. Oleic acid at a ratio 1/10 is added. In this preparation there is a high number of hydrophilic dimeric complexes “free” de-VDBP molecules that, once internalized, will interact through hydrophilic interactions with p53 inhibiting its action. In order to favor internalization without compromising hydrophilia, oleic acid is added at a low ratio, that is 1/10. The trimeric complexes [vitamin D₃/de-VDBP/oleic acid] will interact with VDR at the plasma membrane and trigger the VDR signaling.

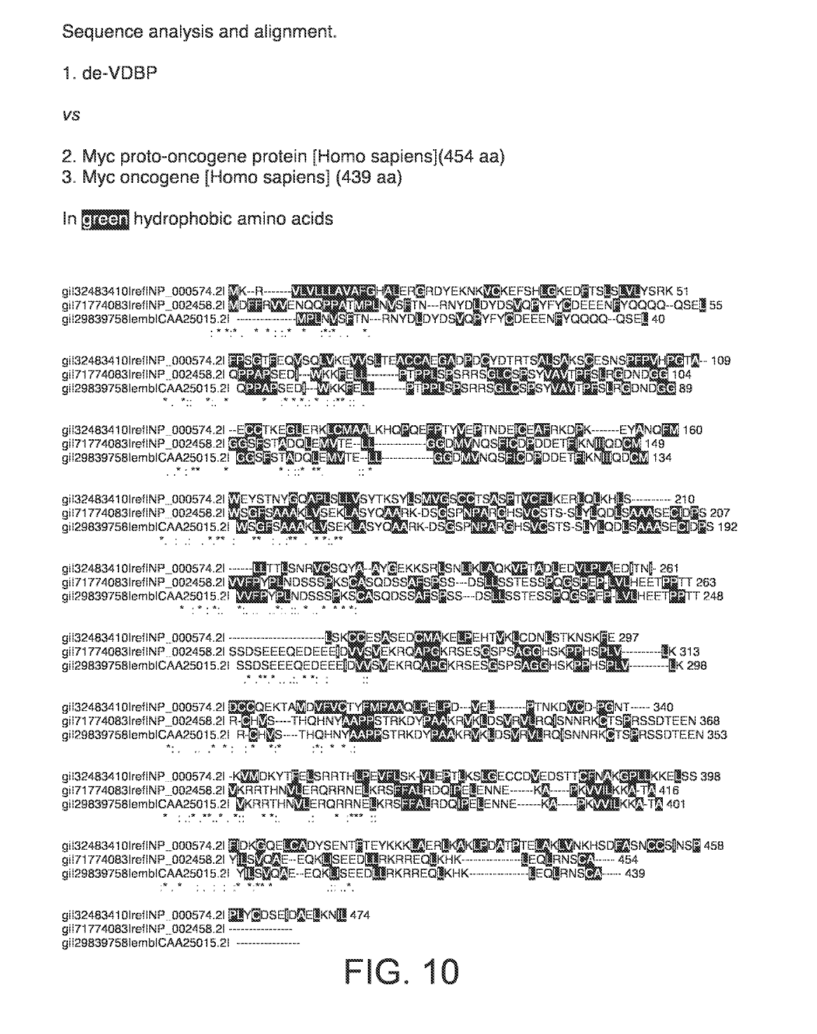

IX-Delta; LS-Delta. A mixture of molecules for melanoma and psoriasis. This mixtures of molecules targets the oncogene product MYC that is involved in human melanomas (Eur J Surg Oncol. 1996 August; 22(4):342-6) and in psoriasis even though psoriasis is not an oncogenic disease (J Invest Dermatol. 1990 November; 95(5 Suppl):7S-9S). As it can be observed in FIG. 10, MYC shows a much less hydrophobic profile than Bcl-2. Therefore, the design of molecules targeted at this protein must take into account hydrophilic interactions involving the GalNAc moiety of de-VDBP. The preparation starts with 1/10 ratio vitaminD₃/de-VDBP. EPA at a ratio 1/10 is added. In this preparation there is a high number of hydrophilic dimeric complexes “free” de-VDBP molecules that, once internalized, will interact through hydrophilic interactions with MYC, thus inhibiting its action. In order to favor internalization without compromising hydrophilia, EPA is added at a low ratio, that is 1/10. The trimeric complexes [vitamin D₃/de-VDBP/EPA] will interact with VDR at the plasma membrane and trigger the VDR signaling. Unlike alpha, beta and gamma, in delta there is EPA instead of oleic acid in order to provide higher flexibility of the molecule to favor the interaction with MYC oncoprotein. Delta can be prepared as an injectable product or as a liposomal preparation for topical use.

IX-Epsilon/IS-Epsilon. A mixture of multimolecular complexes for prostate cancer. Since p53, MYC and Bcl-2 are all involved in the progression of human prostate cancer (Arch Biochem Biophys. 2009 Jun. 15; 486(2):95-102) a mixture of multimolecular complexes with intermediate hydrophobic/hydrophilic characteristics has to be designed. The preparation starts with 5/10 ratio vitaminD₃/de-VDBP. EPA at a ratio 1/1 is added. For best results, a combination with IS-Epsilon is envisaged at a final ration 50% v/v.

Description of examples of multimolecular complexes and mixtures of complexes intended as supplements or for administration by injection targeted to different types of diseases or conditions.

LS-Zeta. A mixture of complexes for autism. Since brain inflammation is one of the hallmarks of autism, multimolecular complexes aimed at this phenomenon were designed. The preparation starts with 1/1 ratio vitaminD₃/de-VDBP. EPA at a ratio 1/1 is added. These ratios provide the highest number of hydrophobic molecules able to cross the blood-brain barrier. The oral bioavailability will favor administration to children. EPA can be substituted for docosahexaenoic acid (DHA), an omega-3 fatty acid that is a primary structural component of the human brain and has proven effective in a number of neurological diseases. The final mixture is: [vitaminD₃/de-VDBP/EPA]+[vitaminD₃/de-VDBP/DHA], 50% v/v.

IX-Eta/IS-Eta; LS-Eta. A mixture of complexes for Myalgic Encephalomyelitis/Chronic Fatigue Syndrome, neurodegenerative disorders, heavy metal-associated neurological conditions. The preparation starts with 5/10 ratio vitaminD₃/de-VDBP. EPA at a ratio 2/10 is added. This preparation can be used in injectable form (IX-Eta) or as liposomal preparation for oral administration (LS-Eta). Also in this case, EPA can be substituted for docosahexaenoic acid (DHA), an omega-3 fatty acid that is a primary structural component of the human brain and has proven effective in a number of neurological diseases. The final mixture is identical to that of 6.2.3.1., but the ratios of UFA are different because of the different permeability of the blood-brain barrier in adults.

IX-Theta/IS-Theta, A mixture of molecules for Chronic Kidney Disease. The preparation starts with 1/1 ratio vitamin D analogue/de-VDBP. EPA at a ratio 5/10 is added.

IX-Iota; IS-Iota; L[X-Iota/S-Iota]. A mixture of molecules for Cardiovascular Diseases. The preparation of IX-Iota starts with 1/1 ratio vitamin D₃/de-VDBP. EPA at a ratio 1/1 is added. Successively, a mixture of IS-iota is added at a ratio 1/1 v/v. The resulting mixture can be prepared also as liposomal preparation for oral administration.

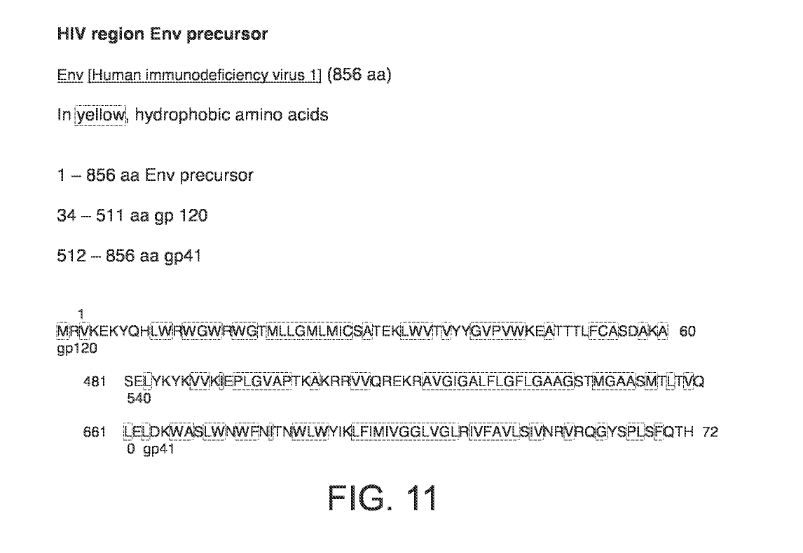

Kappa series. A mixture of molecules targeting the human immunodeficiency virus (HIV). The amino acid sequence of HIV is represented in FIG. 11. In this case, there is no alignment with the sequence of de-VDBP because there is no homology between the two sequences. Looking at the sequence of HIV it is clearly evident that there are long stretches of hydrophobic amino acids where several molecules prepared according to the methods described in 6.1. can interact. Therefore, molecules of the Kappa series were designed according to the concept of producing a mixture of multimolecular complexes with each complex showing a different degree of hydrophobicity and flexibility to adapt to the roundish structure of the virion. Each complex composing the Kappa series is termed with a consecutive number.

Kappa-1. 1/10 ratio vitamin D₃/de-VDBP. EPA at a ratio 1/1 is added.

Kappa-2. 1/1 ratio vitamin D₃/de-VDBP. EPA at a ratio 1/10 is added.

Kappa-3. 1/10 ratio vitamin D₃/de-VDBP. Oleic acid at a ratio 1/1 is added.

Kappa-4. 1/1 ratio vitamin D₃/de-VDBP. Oleic acid at a ratio 1/10 is added.

The final Kappa preparation contains each complex of the Kappa series in equal amount that is 25% v/v. The Kappa series preparation can be formulated also as a liposomal preparation for oral administration.

Molecules and mixtures specifically tailored on the individual VDR genotype. It is well assessed that VDR gene polymorphisms are associated with differential responses to vitamin D₃. Because of this, when vitamin D₃ supplementation is required, determination of VDR genotype is recommended in order to provide the most efficient dose and frequency of administration of vitamin D₃ (Kidney Int. 2009 November; 76(9):931-3). For example, it is well known that the BB and the ff homozygous genotypes are poor responders to vitamin D₃. Specific molecules tailored on the individual VDR genotype take into account the sequence of the VDR and are designed to provide the best interaction with the polymorphic receptor. For example, the Fok-I polymorphism, involves a VDR protein that is 3 amino acids longer. This renders the VDR molecule “stiffer” and as such, with less capability to interact with other proteins and DNA. Such a characteristics of the VDR molecule can be targeted by using poly-unsaturated fatty acids with high flexibility in the molecules described above. For example, α-Linolenic acid or docosahexaenoic acid (DHA) could be used instead of oleic acid or EPA. In individual with known VDR genotype, the type of UFA could be adjusted for any of the molecules described in the section 6.2.2. and 6.2.3.

The complexes of the present invention can be formulated for intra/peri-tumoral injection to treat cancers. For example, a multimolecular (pentameric) mixture of 1/1; 1/0.5; 1/0.2; 1/0.1 ratio of de-VDBP/oleic acid dissolved in different ethanol (20% v/v) and propylene glycol (30% v/v) concentrations according to the following scheme:

1. Ratio 1/1 volume 2.42 mL yields 400 ng/mL deVDBP and 1.97 ng/mL Oleic acid 9.1% ethanol (20% v/v) and propylene glycol (30% v/v).

2. Ratio 1/0.5 volume 2.42 mL yields 400 ng/mL deVDBP and 1.08 ng/mL Oleic acid 9.1% ethanol (20% v/v) and propylene glycol (30% v/v).

3. Ratio 1/0.2 volume 2.42 mL yields 400 ng/mL deVDBP and 0.44 ng/mL Oleic acid 6.4% ethanol (20% v/v) and propylene glycol (30% v/v).

4. Ratio 1/0.1 volume 2.42 mL yields 400 ng/mL deVDBP and 0.19 ng/mL Oleic acid in saline.

5. Ratio 1/0 volume 2.42 mL yields 400 ng/mL deVDBP in saline.

This mixture may be administered using ultrasound-guided peri/intratumoural injection. In this connection, it exploits the well-known anti-cancer properties of oleic acid conjugated to proteins whose folding is influenced by the binding with the fatty acid (FEBS J. 2013 April; 280(8):1733-49). Thus, oleic acid bound to proteins, such as HAMLET (human α-lactalbumin made lethal to tumour cells) or VDBP, induces the apoptosis of cancer cells by exploiting unifying features of cancer cells such as oncogene addiction or the Warburg effect (Oncogene. 2011 Dec. 1; 30(48):4765-79).

This mixture has been found to be particularly effective in cancer patients who are following a nutritional plan consisting in an equi-caloric diet containing 10% or less of carbohydrates, 25% of fats (in particular oleic acid and other UFA), and 50-60% proteins as demonstrated in (Cancer Res. 2011 Jul. 1; 71(13):4484-93). Its effectiveness operates on a variety of cancers since it targets the basic molecular mechanisms of neoplastic transformation (Oncogene. 2011 Dec. 1; 30(48):4765-79). In addition, due to its physico-chemical features, i.e. the formation of molecular complexes resonating in the same range of diagnostic ultrasounds (Brain Stimul. 2013 May; 6(3):409-15), the mixture can be targeted toward the lesion through the mechanical forces exerted by focused ultrasounds. In other words, an ultrasound beam targeted toward the tumor immediately after the injection of the mixture will force the molecules toward the lesion where they will be dissociated by the release of mechanical energy, therefore dramatically increasing their therapeutic efficacy.

We have also found that the mixture is particularly effective when administered as an aerosol with a common nebulizer. Its peculiar multimolecular configuration renders it very effective in stimulating alveolar macrophages, the key elements of the innate immune response against pathogenic viruses, bacteria, micro-organisms and cancer cells (Gerontology. 2013; 59(6):481-9). In fact, it has been demonstrated that emulsions containing oleic acid have the characteristic of being rapidly absorbed through the intranasal route, and show excellent pharmacokinetic properties (Xenobiotica. 2011 July; 41(7):567-77).

Such aerosol administration can be useful in a variety of lung-associated pathologic conditions in addition to primary and metastatic lung cancer such as chronic obstructive pulmonary disease as well as viral or bacterial pneumonias.

Supporting Observations

Invitro Activity:

The biological activity of the molecules and the multimolecular complexes described in the preceding sections was tested in an in vitro system (MCF-7 human breast cancer cell cultures) that is routinely used to assess the biological activity of vitamin D₃ and of the other components of the vitamin D axis (Evid Based Complement Alternat Med. 2012; 2012:310872. Oncol Rep. 2012 December; 28(6):2131-7. Anticancer Res. 2012 March; 32(3):739-46). Human breast carcinoma cells MCF-7 were challenged with different concentration of vitamin D₃, de-VDBP, and [vitamin D₃/de-VDBP/oleic acid]. The results reported in Table 1 clearly show that the novel complexes described in the preceding sections have a much greater biological activity in comparison with vitamin D₃ or de-VDBP. Maximal inhibition of MCF-7 proliferation was achieved with 1 uM vitamin D₃, a very high, non-physiological concentration. The same level of inhibition, however, could be achieved with 1000 fold less vitamin D₃ when it was complexed with de-VDBP and oleic acid.

TABLE 1
Effects of vitamin D₃ and other compounds on MCF-7 human
breast cancer cell proliferation

| | Absorbance units |
|---|---|
| Treatment | (×10³) |
| Control (no addition) | 450 ± 21 |
| Vitamin D₃ 1 nM | 425 ± 10 |
| Vitamin D₃ 1 uM | 55 ± 10 |
| de-VDBP 1 ng/ml | 395 ± 37 |
| [Vitamin D₃ 1 nM/de-VDBP 1 ng/ml/oleic acid 1/1] | 55 ± 9 |

Cell line. MCF-7 cells were obtained from ATCC. Cells were routinely maintained at 37° C. in a humidified atmosphere of 5% CO2 in Eagle's minimum essential medium in Earle's Balanced salt solution, supplemented with 1 mM sodium pyruvate, 10% foetal bovine serum (FBS), 100 U/ml penicillin, and 100 μg/ml streptomycin (Invitrogen, Carlsbad, CA, USA).
Cell proliferation. Assessment of cell proliferation was determined by Calbiochem Rapid Cell Proliferation Kit. Each condition was replicated in quadruplicate samples and each experiment was replicated three times.

In Vivo Activity: Patient a

The molecules described in 6.2.2.3 (IX-Gamma/IS-Gamma) were used as part of the so-called “compassionate approach” in an incurable breast cancer patients who was not eligible for any other conventional therapy. IS-Gamma, 880 ng, was injected into the solid tumour that was located in the proximity of the left axilla. The activation of the immune system and the subsequent anticancer effects were monitored by assessing the blood flow in the tumour. Thus, it is well known that activated macrophages release nitric oxide (NO), a compound that causes vasodilatation and shows powerful anti-cancer properties (J Exp Med. 1989 May 1; 169(5):1543-55. Curr Opin Immunol. 1991 February; 3(1):65-70.). The intratumoural blood flow was monitored with an ultrasound system using the echo-colour-doppler technique. FIG. 12, shows the blood flow inside the solid tumour before injection of IS-Gamma (880 ng) (left panel) and 2 min after the injection. The increase in blood flow in the same area of the tumour, identified by the circular area of necrosis in the centre is dramatic.

In Vivo Activity: Patient B

In a patient with metastatic prostate cancer, IS-Gamma, 880 ng, was injected into the area of the inguinal nodes (440 ng in each side) with the goal of targeting the intraperitoneal, periaortic nodes that a previous MRI had shown being metastatic. FIG. 13, shows that after 3 days of daily injections, the node taken as reference showed a volume decrease of 25%. The node before the treatment is shown in the left panel; the right panel shows the node 3 after 3 days of treatment. The blood vessel was taken as reference in order to make sure that the measurement was accurate and reliable.

In Vivo Activity: Clinical Results

The effectiveness of the complexes according to the present invention is discussed in Emma Ward et al, American Journal of Immunology 10(1); 23-32 2014, the disclosures of which are incorporated herein by reference.

In summary, the present invention provides a method of producing improved supplements based on vitamin D₃ and vitamin D analogues (together termed, Vit Ds). These are stabilized by interaction with de-glycosylated vitamin D-binding protein (de-VDBP). VDBP may be de-glycosylated at position Thr 420 by treatment with sialidase and beta-galactosidase. de-VDBP is used as backbone to stabilize Vit Ds and allow a more efficient interaction with the cellular plasma membrane and with the vitamin D receptor (VDR). Stabilisation is achieved by specific hydrophobic interaction between Vit Ds and 23 hydrophobic amino acids of de-VDBP. The dimeric complex [Vit Ds/de-VDBP] with de-VDBP as backbone may be further stabilised by hydrophobic interaction between a stretch of hydrophobic amino acids in domain III of de-VDBP and unsaturated fatty acids (UFA). The resulting trimeric complex [Vit Ds/de-VDBP/UFA] can be demonstrated to interact with the VDR at the level of the plasma membrane. The resulting tetrameric complex [Vit Ds/de-VDBP/UFA/VDR] is spontaneously internalized into the target cells by cellular transport proteins and, once inside the cell, the protein/protein interaction further strengthened by base-acid interaction between alpha-N-galactosamine at position Thr 420 of de-VDBP backbone and a stretch of acidic amino acids in position 207-215 of VDR. Administration of the stabilised trimeric complex [Vit Ds/de-VDBP/UFA] to normal and pathologic cell cultures evokes cellular responses 10-100 fold greater than those observed with vitamin D₃ alone or with de-VDBP alone. Potency and bio-availability of de-VDBP and of the complexes can be further increased by dissolving the complex in an aqueous alcoholic saline solvent. The trimeric complexes [Vit Ds/de-VDBP/UFA] may be encapsulated in liposomes with different phospholipid compositions in order to produce compounds readily absorbable through the oral route and/or for topical use, e.g. in an ointment. All the stabilised complexes may also be administered sublingually. This strategy enables the production of a family of compounds associated with the backbone of de-VDBP where vitamin D₃ can be substituted for by, for example, non-hypercalcemic VDR agonists (such as eldecalcitol). This family of novel, stabilised complexes of Vit Ds/de-VDBP/UFA can be used in all those conditions where supplementation of vitamin D has proven effective including, but not limited to: prevention of all-cause mortality; stimulation of the immune system; bone health; cardiovascular diseases; cancer; chronic kidney disease, HIV infection; neurodegenerative diseases (Parkinson's, Alzheimer's, autism, myalgic encephalomyelitis, multiple sclerosis), prevention of heavy metal-associated neurological conditions.

1-10. (canceled)

11. A vitamin D-based preparation being a trimeric complex of, as backbone, a de-glycosylated vitamin D-binding protein, vitamin D₃ or an analog thereof, and at least one unsaturated fatty acid.

12. A preparation according to claim 11 further including an aqueous alcoholic saline solvent.

13. A preparation according to claim 11 wherein the unsaturated fatty acid is oleic acid or eicosapentaenoic acid.

14. A preparation according to claim 12 wherein the unsaturated fatty acid is oleic acid or eicosapentaenoic acid.

15. An orally administrable composition comprising the preparation according to claim 11 encapsulated in liposomes.

16. An orally administrable composition comprising the preparation according to claim 12 encapsulated in liposomes.

17. An orally administrable composition comprising the preparation according to claim 13 encapsulated in liposomes.

18. A composition formulated for administration by nebulization and comprising the preparation according to claim 11.

19. A composition formulated for administration by nebulization and comprising the preparation according to claim 12.

20. A composition formulated for administration by nebulization and comprising the preparation according to claim 13.

21. A composition formulated for administration as a suppository and comprising the preparation according to claim 11.

22. A composition formulated for administration as a suppository and comprising the preparation according to claim 12.

23. A composition formulated for administration as a suppository and comprising the preparation according to claim 13.

24. A composition formulated for administration as an enema and comprising the preparation according to claim 11.

25. A composition formulated for administration as an enema and comprising the preparation according to claim 12.

26. A composition formulated for administration as an enema and comprising the preparation according to claim 13.

27. A method for producing the preparation according to claim 11 comprising sequentially de-glycosylating a vitamin D-binding protein to obtain said de-glycosylated vitamin D-binding protein, complexing the de-glycosylated protein with Vitamin D₃ or an analog thereof, and further complexing a dimeric complex so obtained with the at least one unsaturated fatty acid.

28. A method for producing the orally administrable composition according to claim 15 comprising encapsulating in liposomes a complex obtained by a method comprising sequentially de-glycosylating a vitamin D-binding protein to obtain said de-glycosylated vitamin D-binding protein, complexing the de-glycosylated protein with Vitamin D₃ or an analog thereof, and further complexing a dimeric complex so obtained with at least one unsaturated fatty acid.

29. A method for producing the orally administrable composition according to claim 16 comprising encapsulating in liposomes a complex obtained by a method comprising sequentially de-glycosylating a vitamin D-binding protein to obtain said de-glycosylated vitamin D-binding protein, complexing the de-glycosylated protein with Vitamin D₃ or an analog thereof, and further complexing a dimeric complex so obtained with at least one unsaturated fatty acid.

30. A method for producing the orally administrable composition according to claim 17 comprising encapsulating in liposomes a complex obtained by a method comprising sequentially de-glycosylating a vitamin D-binding protein to obtain said de-glycosylated vitamin D-binding protein, complexing the de-glycosylated protein with Vitamin D₃ or an analog thereof, and further complexing a dimeric complex so obtained with at least one unsaturated fatty acid.

31. A method of treating a patient suffering from cancer by administration of a therapeutically effective amount of the preparation according to claim 11.

32. A method of treating a patient suffering from cancer by administration of a therapeutically effective amount of the preparation according to claim 12.

33. A method of treating a patient suffering from cancer by administration of a therapeutically effective amount of the preparation according to claim 13.


SEQUENCE LISTING (13 sequences)

SEQ ID NO: 1 - protein, 474 aa, Homo sapiens
  vitamin D-binding protein isoform 1
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 2 - protein, 352 aa, Homo sapiens
  vitamin D-binding protein isoform 2
MAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPKELAKLVNKRSDFASNCCSINSPPLYCDSEIDAELKNIL

SEQ ID NO: 3 - protein, 493 aa, Homo sapiens
  vitamin D-binding protein isoform 3
MLWSWSEERGGAARLSGRKMKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 4 - protein, 427 aa, Homo sapiens
  vitamin D receptor
MEAMAASTSLPDPGDFDRNVPRICGVCGDRATGFHFNAMTCEGCKGFFRRSMKRKALFTCPFNGDCRITKDNRRHCQACRLKRCVDIGMMKEFILTDEEVQRKREMILKRKEEEALKDSLRPKLSEEQQRIIAILLDAHHKTYDPTYSDFCQFRPPVRVNDGGGSHPSRPNSRHTPSFSGDSSSSCSDHCITSSDMMDSSSFSNLDLSEEDSDDPSVTLELSQLSMLPHLADLVSYSIQKVIGFAKMIPGFRDLTSEDQIVLLKSSAIEVIMLRSNESFTMDDMSWTCGNQDYKYRVSDVTKAGHSLELIEPLIKFQVGLKKLNLHEEEHVLLMAICIVSPDRPGVQDAALIEAIQDRLSNTLQTYIRCRHPPPGSHLLYAKMIQKLADLRSLNEEHSKQYRCLSFQPECSMKLTPLVLEVFGNEIS

SEQ ID NO: 5 - protein, 23 aa, Homo sapiens
  23 hydrophobic amino acids near the amino terminus of VDBP
MKRVLVLLLAVAFGHALERGRDY

SEQ ID NO: 6 - protein, 23 aa, Homo sapiens
  23 amino acids near the carboxyl terminus of the VDR
SFQPECSMKLTPLVLEVFGNEIS

SEQ ID NO: 7 - protein, 28 aa, Homo sapiens
  de-VDBP sequence binding EPA
DSTTCFNAKGPLLKKELSSFIDKGQELC

SEQ ID NO: 8 - protein, 14 aa, Homo sapiens
  hydrophobic amino acid sequence near the carboxyl terminus of VDR
LTPLVLEVFGNEIS

SEQ ID NO: 9 - protein, 232 aa, Homo sapiens
  B-cellCLL/lymphoma 3
MAHAGRTGYDNREIVMKYIHYKLSQRGYEGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDSWLSLKTLLSLALVGACITLGAYLGHK

SEQ ID NO: 10 - protein, 393 aa, Homo sapiens [sequence omitted]

SEQ ID NO: 11 - protein, 454 aa, Homo sapiens
  Myc proto-oncogene protein
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 12 - protein, 439 aa, Homo sapiens
  Myc oncogene
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 13 - protein, 180 aa, Human immunodeficiency virus 1
  Env
MRVKEKYQHLWRWGWRWGTMLLGMLMICSATEKLWVTVYYGVPVWKEATTTLFCASDAKASELYKYKVVKIEPLGVAPTKAKRRVVQREKRAVGIGALFLGFLGAAGSTMGAASMTLTVQLELDKWASLWNWFNITNWLWYIKLFIMIVGGLVGLRIVFAVLSIVNRVRQGYSPLSFQTH